(19) United States
(12) Patent Application Publication
(10) Pub. No.: US 2025/0034087 A1
(43) Pub. Date: Jan. 30, 2025

(54) PROCESS FOR PRODUCTION OF VITAMIN A

(71) Applicant: DSM IP ASSETS B.V., Heerlen (NL)

(72) Inventors: Werner BONRATH, Kaiseraugst (CH); Jonathan Alan MEDLOCK, Kaiseraugst (CH); Marc-André MUELLER, Kaiseraugst (CH); Marcel STETTLER, Kaiseraugst (CH); Bettina WUESTENBERG, Kaiseraugst (CH); Viktor ZIMMERMANN, Kaiseraugst (CH)

(21) Appl. No.: 18/712,069

(22) PCT Filed: Nov. 23, 2022
(86) PCT No.: PCT/EP2022/082915

§ 371 Date: May 21, 2024

(30) Foreign Application Priority Data
Nov. 26, 2021 (EP) 21210683.5

(51) Int. Cl. C07C 403/20; C07D 333/48

(52) U.S. Cl. CPC C07C 403/20; C07D 333/48; C07C 2601/16

(57) ABSTRACT

The present invention relates to a new process for the production of vitamin A and/or its derivatives.

The present invention relates to a new process for the production of vitamin A and/or its derivatives.

Vitamin A or its derivatives (such as vitamin A acetate, vitamin A propionate or vitamin A palmitate)

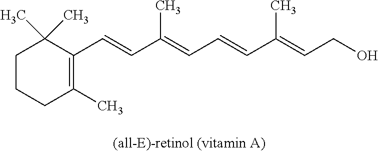 [SMILES: CC1=C(/C=C/C(C)=C/C=C/C(C)=C/CO)C(C)(C)CCC1]

is an important ingredient for many applications. Vitamin A plays a role in a variety of functions throughout the body, such as e.g., vision process, gene transcription, immune function, bone metabolism, haematopoiesis, skin and cellular health and antioxidant function.

Due to the importance of vitamin A (and its derivatives) and the complexity of the synthesis thereof, there is always a need for improved processes of production.

The goal of the present invention was to find a new synthesis of vitamin A or its derivates. The aim was achieved by the synthetic route of the present invention.

The new synthesis how to obtain vitamin A and/or its derivatives can be seen from the following scheme:

The following scheme shows how vitamin A (or derivatives thereof), which is the compound of formula (I), can be obtained:

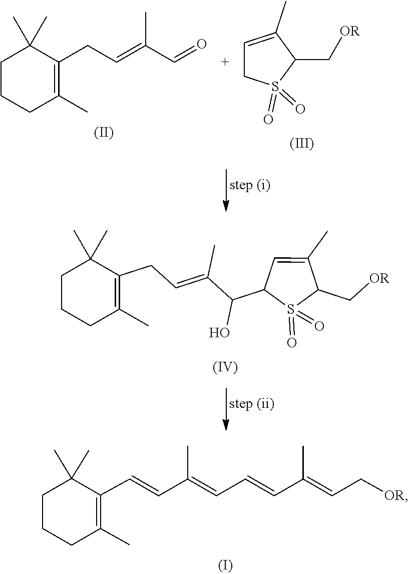

wherein R is H or

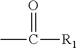

and wherein R₁ is a linear or branched C₁-C₁₈ alkyl moiety.

In context of the present patent application the carbon-carbon double bonds of the compounds shown in the patent application can have any E-Z configuration. The E-Z configuration is not an essential feature of the present invention.

The new process consists of 2 steps (step (i) and step (ii)).

The compound of formula (II) can be produced according to any process known from the prior art.

The other starting material, which is the compound of formula (III), can be produced by reacting a compound of formula (VI)

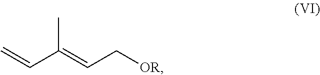

wherein R and R₁ have the same meaning as defined for the compound of formula (III), with SO₂ in an inert solvent.

It is known from the prior art how to obtain the compounds of formula (VI) (e. g. from Z. Wu et al, J. Am. Chem. Soc., 2005, 17433).

Therefore, the present invention relates to a process (P) for the production of a compound of formula (I)

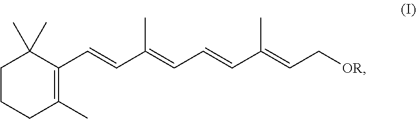

wherein

### R is H or

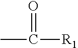

and wherein R₁ is a linear or branched C₁-C₁₈ alkyl moiety, characterized in that in a first step (step (i))
the compound of formula (II)

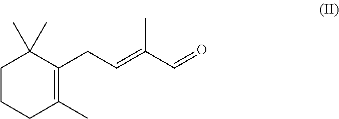

is reacted with a compound of formula (III)

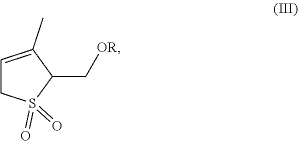

wherein R (and also R₁) has the same meanings as defined for the compound of formula (I), and
then in a second step (step (ii))
the reaction product of step (i), which is the compound of formula (IV)

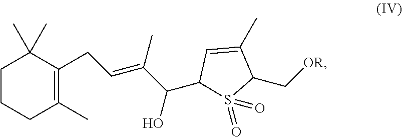

wherein R (and also R₁) has the same meanings as defined for the compound of formula (I),
is converted into the compound of formula (I) by heating the reaction mixture.

Preferably in the compound of formula (I)

### R is H or

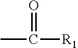

and wherein R₁ is a —CH₃, —CH₂CH₃ or —(CH₂)₁₄CH₃.

More preferably, R is

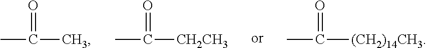

Most preferably, R is

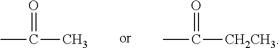

Therefore, the present invention relates to a process (P1), which is process (P), wherein R is H or

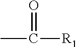

and wherein R₁ is a —CH₃, —CH₂CH₃ or —(CH₂)₁₄CH₃.

Therefore, the present invention relates to a process (P1′), which is process (P), wherein R is

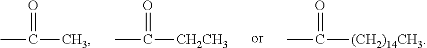

Therefore, the present invention relates to a process (P1″), which is process (P), wherein R is

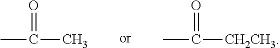

In the following the two steps will be discussed in more details.

### Step (i)

In the first step according to the present invention a compound of formula (II), which is 2-methyl-4-(2,6,6-trimethylcyclohex-1-en-1-yl)but-2-enal (also known as boronal) is reacted with a compound of formula (III), wherein R is H

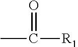

and wherein R₁ is a linear or branched C₁-C₁₈ alkyl moiety,
to form the compound of formula (IV), wherein R₁ has the same meanings as defined for compound of formula (III):

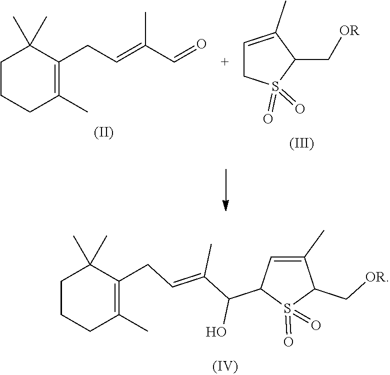

The process of step (i) is usually carried out in at least one inert solvent.

The solvent is usually a polar aprotic or a non-polar aprotic solvent.

Suitable solvents are e.g., THF, diethyl ether, cyclopentyl methyl ether, tert-butyl methyl ether, MeTHF, toluene, heptane and n-hexane.

Therefore, the present invention relates to a process (P2), which is process (P), (P1′), (P1′) or (P1″), wherein the reaction of step (i) is carried out in at least one inert solvent.

Therefore, the present invention relates to a process (P2′), which is process (P2), wherein the at least one inert solvent is a polar aprotic or a non-polar aprotic solvent.

Therefore, the present invention relates to a process (P2″), which is process (P2), wherein the at least one inert solvent is chosen from the group consisting of THF, diethyl ether, cyclopentyl methyl ether, tert-butyl methyl ether, MeTHF, toluene, heptane and n-hexane.

Furthermore, the reaction of step (i) is carried out in the presence of at least one strong base. The pK_{B} value of strong bases is below 2.

Suitable bases are for example NaH, n-BuLi, tert-BuLi, sec-BuLi, LiHMDS, NaHMDS, LDA, NaDA, NaNH₂, KNH₂, LiNH₂, organic amine bases, NaNH₂, LiNH₂, as well as also the analogous potassium salts of these bases.

The strong base in step (i) is usually used in an amount of 0.8 to 2.5 mol-equivalent in regard to the compound of formula (III).

Therefore, the present invention relates to a process (P3), which is process (P), (P1′), (P1′), (P1″), (P2), (P2′) or (P2″), wherein the reaction of step (i) is carried out in the presence of at least one strong base.

Therefore, the present invention relates to a process (P3′), which is process (P3), wherein the pK_{B} value of strong base (or mixture of bases) is below 2.

Therefore, the present invention relates to a process (P3″), which is process (P2), wherein the at least one strong base is chosen from the group consisting of NaH, n-BuLi, tert-BuLi, sec-BuLi, LiHMDS, NaHMDS, LDA, NaDA, NaNH₂, KNH₂, LiNH₂, organic amine bases, NaNH₂, LiNH₂ and the analogous potassium salts of these bases.

Therefore, the present invention relates to a process (P3′″), which is process (P3), (P3′) or (P3″), wherein the at least one strong base is used in an amount of 0.8 to 2.5 mol-equivalent in regard to the compound of formula (III).

Furthermore, the reaction of step (i) is carried out at low temperatures.

Preferably, the reaction of step (i) is carried out at a temperature of −90° C. to 25° C.

More preferably, the reaction of step (i) is carried out at a temperature of −80° C. to 5° C.

Therefore, the present invention relates to a process (P4), which is process (P), (P1′), (P1′), (P1″), (P2), (P2′), (P2″), (P3), (P3′), (P3″) or (P3′″), wherein the reaction of step (i) is carried at low temperatures.

Therefore, the present invention relates to a process (P4′), which is process (P4), wherein the reaction of step (i) is carried out at a temperature of −90° C. to 25° C.

Therefore, the present invention relates to a process (P4″), which is process (P4), wherein the reaction of step (i) is carried out at a temperature of −80° C. to 5° C.

Furthermore, usually the compounds of formula (II) and of formula (III) are used in the reaction of step (i) in a molar ratio of 0.8:1 to 1:0.8.

Therefore, the present invention relates to a process (P5), which is process (P), (P1′), (P1′), (P1″), (P2), (P2′), (P2″), (P3), (P3′), (P3″), (P3′″), (P4), (P4′) or (P4″), wherein the compounds of formula (II) and of formula (III) are used in the reaction of step (i) in a molar ratio of 0.8:1 to 1:0.8.

At the end of the reaction of step (i), which is usually carried out for a period of several minutes to several hours, the reaction product of formula (IV) can be isolated (and optionally purified) and then used in step (ii).

But it is also possible to use the reaction mixture of step (i) as such in step (ii).

### Step (ii)

In the second step according to the present invention the compound of formula (IV)

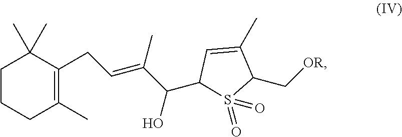

wherein R (and also R₁) has the same meanings as defined for the compound of formula (III), is dissolved in at least one inert solvent.

Usually and preferably, the inert solvent is a polar aprotic or a non-polar aprotic solvent.

In case the reaction mixture of step (i) as such is used in step (ii) there is no need to add a solvent (however it is also possible to add a solvent in this case).

Suitable solvents are e.g. THF, diethyl ether, cyclopentyl methyl ether, tert-butyl methyl ether, MeTHF, toluene, heptane and n-hexane.

Therefore, the present invention relates to a process (P6), which is process (P), (P1′), (P1′), (P1″), (P2), (P2′), (P2″), (P3), (P3′), (P3″), (P3′″), (P4), (P4′), (P4″) or (P5), wherein step (ii) the reaction product of step (i), which is the compound of formula (IV)

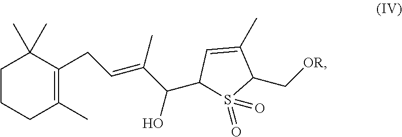

wherein R (and also R₁) has the same meanings as defined for the compound of formula (III), is dissolved in at least one inert solvent.

Therefore, the present invention relates to a process (P6′), which is process (P6), wherein the at least one inert solvent is a polar aprotic or a non-polar aprotic solvent.

Therefore, the present invention relates to a process (P6″), which is process (P6) or (P5′), wherein the at least one inert solvent is chosen from the group consisting of THF, diethyl ether, cyclopentyl methyl ether, tert-butyl methyl ether, MeTHF, toluene, heptane and n-hexane.

The reaction of step (ii) is carried out at an elevated temperature.

Usually and preferably the reaction of step (ii) is carried out a temperature of 40° C. to 120° C.

Therefore, the present invention relates to a process (P7), which is process (P), (P1′), (P1′), (P1″), (P2), (P2′), (P2″), (P3), (P3′), (P3″), (P3′″), (P4), (P4′), (P4″), (P5), (P6), (P6′) or (P6″), wherein the reaction of step (ii) is carried out at an elevated temperature.

Therefore, the present invention relates to a process (P7′), which is process (P7), wherein the reaction of step (ii) is carried out a temperature of 40° C. to 120° C.

Step (ii) can be carried out in the presence or absence of at least one nitrogen containing base.

Therefore, the present invention relates to a process (P8), which is process (P), (P1′), (P1′), (P1″), (P2), (P2′), (P2″), (P3), (P3′), (P3″), (P3′″), (P4), (P4′), (P4″), (P5), (P6), (P6′), (P6″), (P7) or (P7′), wherein the reaction of step (ii) is carried out in the presence a nitrogen containing base.

Therefore, the present invention relates to a process (P9), which is process (P), (P1′), (P1′), (P1″), (P2), (P2′), (P2″), (P3), (P3′), (P3″), (P3′″), (P4), (P4′), (P4″), (P5), (P6), (P6′), (P6″), (P7) or (P7′), wherein the reaction of step (ii) is carried out in the absence of a nitrogen containing base.

The reaction of step (ii) is usually carried out for several hours.

At the end of the reaction of step (ii) the reaction product of step (ii), which is the compound of formula (I) can be isolated and purified by commonly known and used methods.

But it is also possible to use the reaction mixture of step (ii) as such.

A further embodiment of the present invention relates to the new compounds, which are those of formula (IV)

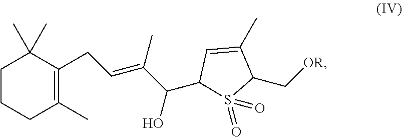

wherein

### R is H or

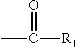

and wherein R₁ is a linear or branched C₁-C₁₈ alkyl moiety.

Therefore, the present invention relates to compounds of formula (IV)

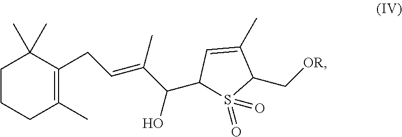

wherein R is H or

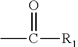

and wherein R₁ is a linear or branched C₁-C₁₈ alkyl moiety.

Preferred are the following compounds of formula (IVa), (IVb), (IVc) and (IVd)

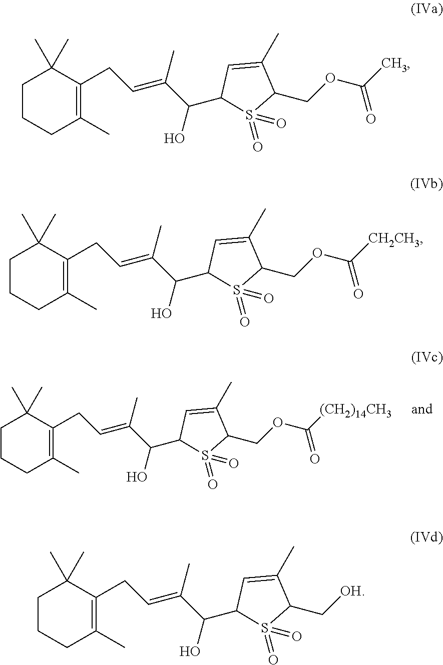

Therefore, the present invention relates to the compounds of formula (IVa), (IVb), (IVc) and (IVd)

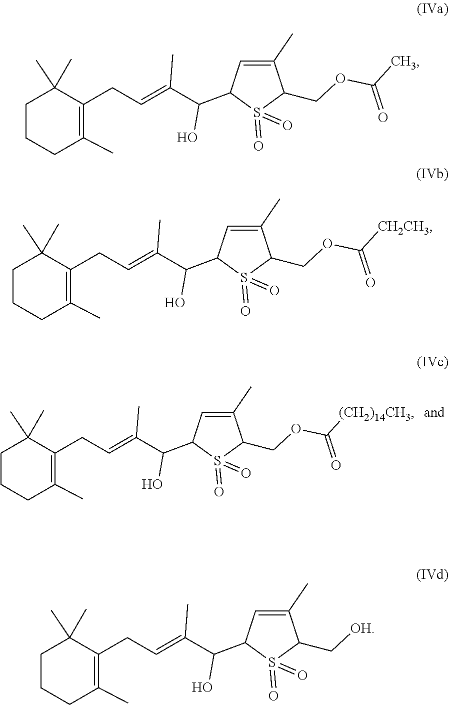

Furthermore, the process of step (ii) can be followed by analytical methods (e.g., NMR) to see the progress of the reaction.

It was observed that in step (ii) two intermediates can be detected:

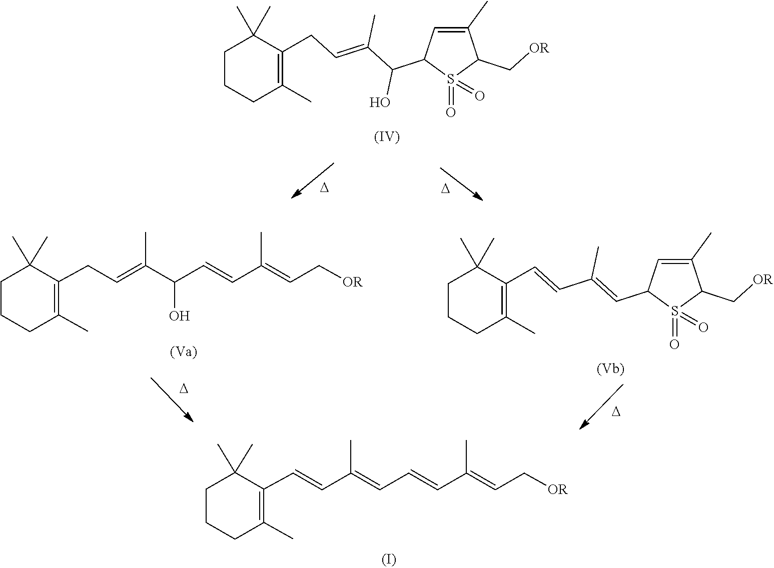

Therefore, the present invention relates to compounds of formula (Vb)

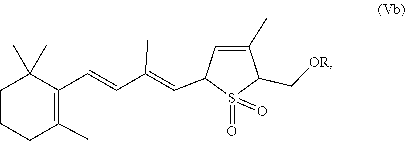

wherein

### R is H or

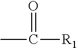

and wherein R₁ is a linear or branched C₁-C₁₃ alkyl moiety.

Therefore, the present invention relates to the compounds of formula (V′b), (V″b), (V′″b) and (V″″b)

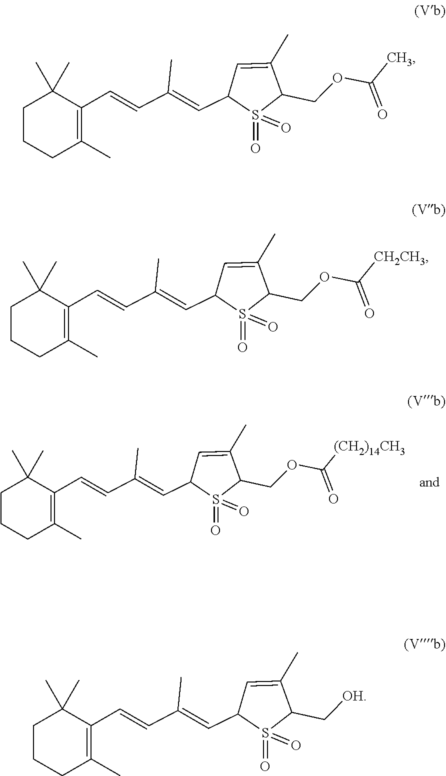

The following example serve to illustrate the invention. The temperature is given in ° C. and all percentages are related to the weight.

### EXAMPLES

### Example 1: Retinyl Acetate Via Compound of Formula (IVa)

In a two-necked round bottom flask C₁₄-aldehyde ((II), (E)-2-methyl-4-(2,6,6-trimethylcyclohex-1-en-1-yl)but-2-enal) (260 mg) and (3-methyl-1,1-dioxido-2,5-dihydrothiophen-2-yl)methyl acetate (268 mg) were dissolved in anhydrous THF (3.75 ml). Under inert gas atmosphere, the reaction mixture was cooled to −75° C. A 2M solution of LDA in THF/n-hexanes (1.25 ml, 2.08 eq.) was added dropwise and stirring was continued for another 10 min at −75° C. The cooling bath was removed and semi-saturated NH₄Cl-solution (12.5 ml) was added. The two-phasic mixture was transferred into a separation funnel and extracted with diethyl ether (2×25 ml). The combined organic extracts were washed subsequently with water (2×12.5 ml) and brine (12.5 ml), filtered and concentrated under reduced pressure (40° C., 5 mbar). The crude product (521 mg) was obtained as a yellow oil. After purification by column chromatography (SiO₂, cyclohexane/ethyl acetate 8:2) 163.3 mg of the compound of formula (IVa)

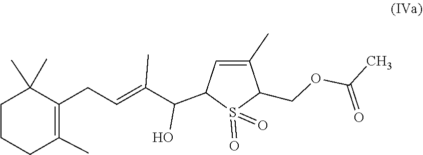 [SMILES: CC(=O)OCC1C(C)=CC(C(O)/C(C)=C/CC2=C(C)CCCC2(C)C)S1(=O)=O]

was obtained.

The oil was placed in a dried two necked round bottom flask and dissolved in toluene (5 mL) under an argon atmosphere. The reaction mixture was heated to reflux for 4 h. All volatiles were evaporated under reduced pressure (40° C., 5 mbar) to obtain retinyl acetate.

### Example 2: Compound of Formula (IVb)

In a two-necked round bottom flask, C₁₄-aldehyde ((II), (E)-2-methyl-4-(2,6,6-trimethylcyclohex-1-en-1-yl)but-2-enal) (260 mg) and (3-methyl-1,1-dioxido-2,5-dihydrothiophen-2-yl)methyl propionate (281 mg) were dissolved in anhydrous THF (3.75 ml). Under inert an acetone/dry-ice cooling bath to −75° C. A 2M solution of LDA in THF/n-hexanes (1.25 ml, 2.08 eq.) was added dropwise and stirring was continued for another 10 min at −75° C. The cooling bath was removed and semi-saturated NH₄Cl-solution (12.5 ml) was added. The two-phasic mixture was transferred into a separation funnel and extracted with diethyl ether (2×25 ml). The combined organic extracts were washed subsequently with water (2×12.5 ml) and brine (12.5 ml), filtered and concentrated under reduced pressure (40° C., 5 mbar). The crude product was obtained as a yellow oil. After purification by column chromatography (SiO₂, cyclohexane/ethyl acetate 8:2) 115 mg of the compound of formula (IVb)

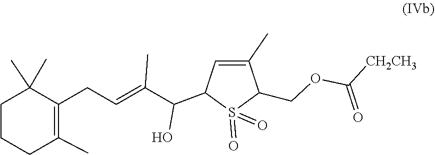 [SMILES: CCC(=O)OCC1C(C)=CC(C(O)/C(C)=C/CC2=C(C)CCCC2(C)C)S1(=O)=O]

was obtained.

1. Process for the production of a compound of formula (formula (I) 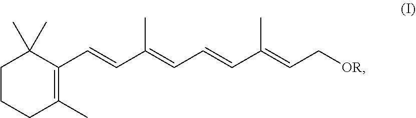
wherein
R is H or 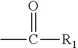
and wherein R₁ is a linear or branched C₁-C₁₈ alkyl moiety, characterized in that in a first step (step (i))
the compound of formula (II) 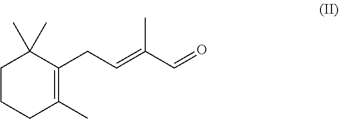
is reacted with a compound of formula (III) 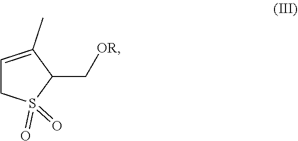
wherein R (and also R₁) has the same meanings as defined for the compound of formula (I), and then in a second step (step (ii))
the reaction product of step (i), which is the compound of formula (IV) 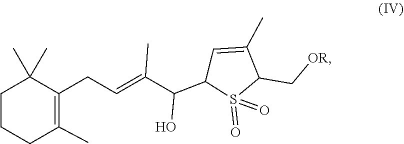
wherein R (and also R₁) has the same meanings as defined for the compound of formula (I), is converted into the compound of formula (I) by heating the reaction mixture.

2. The process according to claim 1, wherein the reaction of step (i) is carried out in at least one inert solvent.

3. The process according to claim 2, wherein the at least one inert solvent is a polar aprotic or a non-polar aprotic solvent.

4. The process according to claim 2, wherein the at least one inert solvent is chosen from the group consisting of cyclopentyl methyl ether, tert-butyl methyl ether, MeTHF, toluene, heptane and n-hexane.

5. The process according to claim 1, wherein the reaction of step (i) is carried out in the presence of at least one strong base.

6. The process according to claim 5, wherein the pK_{B} value of strong base (or mixture of bases) is below 2.

7. The process according to claim 5, wherein the at least one strong base is used in an amount of 0.8 to 2.5 mol-equivalent in regard to the compound of formula (III).

8. The process according to claim 1, wherein the reaction of step (i) is carried out at a temperature of −90° C. to 25° C.

9. The process according to claim 1, wherein, step (ii) the reaction product of step (i), which is the compound of formula (IV) 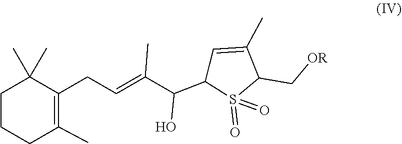
wherein R has the same meanings as defined for compound of formula (I), is dissolved in at least one inert solvent.

10. The process according to claim 9, wherein the at least one inert solvent is a polar aprotic or a non-polar aprotic solvent.

11. The process according to claim 9, wherein the at least one inert solvent is chosen from the group consisting of THF, diethyl ether, cyclopentyl methyl ether, tert-butyl methyl ether, MeTHF, toluene, heptane and n-hexane.

12. The process according to claim 1, wherein the reaction of step (ii) is carried out at an elevated temperature.

13. The process according to claim 12, wherein the reaction of step (ii) is carried out at a temperature of 40° C. to 120° C.

14. Compounds of formula (IV) 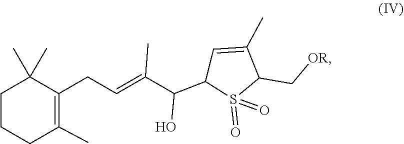
wherein R is H or 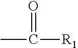
and wherein R₁ is a linear or branched C₁-C₁₈ alkyl moiety.

15. Compounds of formula (Vb) 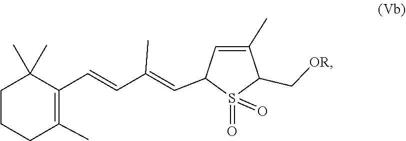
wherein
R is H or 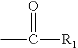
and wherein R₁ is a linear or branched C₁-C₁₈ alkyl moiety.
